(19) United States
(12) Patent Application Publication
(10) Pub. No.: US 2023/0355532 A1
(43) Pub. Date: Nov. 9, 2023

(54) ORAL DRUG DELIVERY DEVICE WITH EXPANDING BAND

(71) Applicant: Eli Lilly and Company, Indianapolis, IN (US)

(72) Inventors: Eric Gustin HASSENPFLUG, Westerville, OH (US); Anthony David DUONG, Columbus, OH (US); Erik William EDWARDS, Gahanna, OH (US); Brian R. FORTNER, New Albany, OH (US); Larry Keith HOOKS, Jr., Columbus, OH (US); Steven HUCKABY, Blacklick, OH (US); Trevor Alan PETREL, Columbus, OH (US); Sarah Louise CLARK, Somerville, MA (US)

(21) Appl. No.: 18/245,425

(22) PCT Filed: Sep. 15, 2021
(86) PCT No.: PCT/US2021/050434

§ 371 Date: Mar. 15, 2023

Related U.S. Application Data
(60) Provisional application No. 63/079,005, filed on Sep. 16, 2020.

(51) Int. Cl. A61K 9/48; A61K 9/70

(52) U.S. Cl. CPC A61K 9/4808; A61K 9/7007; A61K 9/4891

(57) ABSTRACT

The present disclosure provides a drug delivery device. The drug delivery device is taken orally by a patient, and a capsule degrades within the gastrointestinal (GI) tract of the patient. An expanding band coated with a drug and located within the capsule expands and interfaces with the interior of the GI tract, allowing the drug to diffuse through the GI tract. After a period of time, the expanding band degrades and passes through the GI tract.

### FIELD OF THE DISCLOSURE

The present disclosure relates to an oral drug delivery device. More specifically, the present disclosure relates to an expanding band coated with a drug and configured to deliver the drug through contact with the interior of a gastrointestinal tract of a patient.

### BACKGROUND OF THE DISCLOSURE

For patients being treated with a drug or some other biologically active compound, it is often most convenient to receive the compound orally. However, the nature of some compounds prevents them from maintaining their activity once consumed. For example, some compounds are denatured, digested, or otherwise deactivated once placed in the environment of the gastrointestinal (GI) system. Additionally, some compounds have low rates of diffusion into the bloodstream from the GI system when passing through, which can prevent adequate dosages from being delivered to the patient. For compounds with these characteristics, patients often receive the compounds through injection, which is painful and inconvenient. Accordingly, it is desirable to develop an oral drug delivery device that can successfully deliver a drug that would otherwise be ineffective when taken orally.

### SUMMARY

The present disclosure provides a drug delivery device. The drug delivery device is taken orally by a patient, and a capsule degrades within the gastrointestinal (GI) tract of the patient. An expanding band coated with a drug and located within the capsule expands and interfaces with the interior of the GI tract, allowing the drug to diffuse through the GI tract. After a period of time, the expanding band degrades and passes through the GI tract.

In an exemplary embodiment, an oral drug delivery device is disclosed including a biodegradable capsule; and an expanding band coiled within the biodegradable capsule, the expanding band including: a substrate; an interfacing surface on the substrate; and a solid drug applied to the interfacing surface; wherein the expanding band is configured to expand after the biodegradable capsule has at least partially degraded, the interfacing surface is configured to interface with an interior of a gastrointestinal tract of a patient, and the expanding band is further configured to degrade within the gastrointestinal tract of the patient.

In another embodiment, an oral drug delivery device is disclosed including an enteric coating; a plurality of substrate pieces within the enteric coating, wherein the plurality substrate pieces are coupled together; an interfacing surface spanning the plurality of substrate pieces, wherein the interfacing surface is configured to interface with an interior of a gastrointestinal tract of a patient; and a drug applied to the interfacing surface, the drug configured to be absorbed through the interior of the gastrointestinal tract of the patient.

In yet another embodiment, an oral drug delivery device is disclosed including a capsule configured to degrade; a substrate coiled within the capsule and configured to at least partially uncoil and degrade after the capsule degrades; an interfacing surface on the substrate, the interfacing surface facing radially outward and configured to interface with an interior of a gastrointestinal tract of a patient; and a drug applied to the interfacing surface and configured to enter a bloodstream of a patient by diffusing through the interior of the gastrointestinal tract of the patient.

In still yet another embodiment, an oral drug delivery device is disclosed including a biodegradable capsule, an expandable band, and a solid drug disposed on the expandable band, wherein the oral drug delivery device has a loaded configuration in which the expandable band is coiled within the capsule, an expanded configuration in which the expandable band at least partially uncoils upon degradation of the capsule to place the solid drug in contact with a patient, and a collapsed configuration in which the expandable band degrades to pass through the patient.

### BRIEF DESCRIPTION OF THE DRAWINGS

The above-mentioned and other features and advantages of this disclosure, and the manner of attaining them, will become more apparent and the invention itself will be better understood by reference to the following description of embodiments of the invention taken in conjunction with the accompanying drawings, wherein:

FIG. 1 is a perspective view of a drug delivery device according to the present disclosure shown in a loaded configuration;

FIG. 2 is a perspective view of an expanding band of the drug delivery device of FIG. 1 shown in an expanded configuration;

FIGS. 3-6 are perspective views of the drug delivery device of FIG. 1 activating within a gastrointestinal tract;

FIGS. 7-8 are perspective views of the expanding band of FIG. 2 comprising various drug application patterns;

FIGS. 9-10 are perspective views of the drug delivery device of FIG. 1 with varying substrate thicknesses;

FIG. 11-12 are perspective views of the expanding band of FIG. 2 with varying substrate pieces; and

FIG. 13 is a perspective view of the expanding band of FIG. 11 partially degraded in a gastrointestinal tract.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

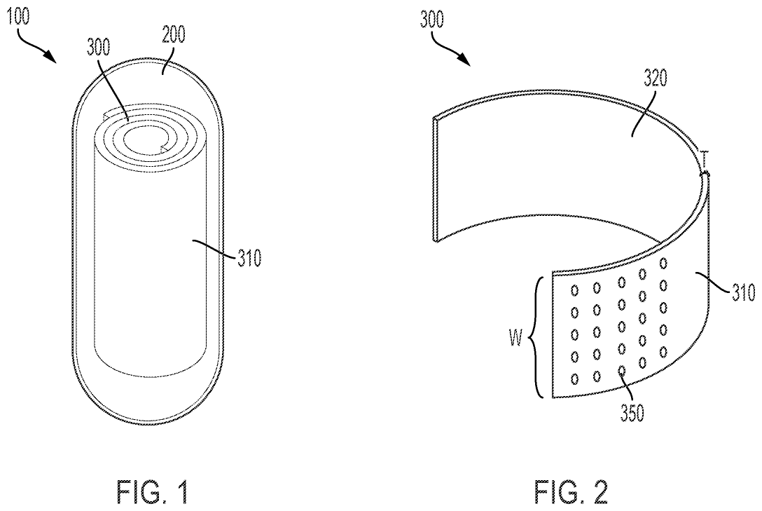

Referring to FIGS. 1-2, a drug delivery device 100 is disclosed. For the purposes of this disclosure, a drug is understood to be any biologically active compound that can be administered to a patient/user. Drug delivery device 100 comprises a capsule 200 and an expanding band 300.

In an exemplary embodiment, capsule 200 is biodegradable and is configured to dissolve or otherwise break down. Capsule 200 is configured to degrade upon a change in the environment of the capsule 200, including changes in pH, temperature, light, chemical concentrations, electric current or voltage, pressure, velocity, acceleration, or any other environmental factor. In the illustrated example, the capsule 200 is configured to degrade when moving from a low pH environment, e.g., the stomach, to a relatively higher pH environment, e.g., the small intestine. Accordingly, capsule 200 may be configured to degrade at various points along the GI tract. Capsule 200 may be composed of a solid housing surrounding expanding band 300. Capsule 200 may be constructed by bringing two portions of the capsule 200 together and coupling the portions through friction, welding, adhesives, or mechanical coupling means. Capsule 200 may also be sprayed onto or otherwise applied as a coating covering expanding band 300. Capsule 200 may include or be coated with an enteric coating configured to remain stable in the stomach but break down in the higher alkaline pH of the intestine. Exemplary enteric coatings include hydroxy methyl cellulose (and other cellulose derivatives) and polyvinyl alcohol, although any suitable enteric coating may be provided.

In the illustrated embodiment, expanding band 300 is composed of multiple layers of materials, including at least a substrate 320, an interfacing surface 310, and a drug 350 disposed on the interfacing surface 310. Expanding band 300 is configured to be coiled within capsule 200 in a loaded configuration (see FIG. 1) and to expand and at least partially uncoil after dissolution of or removal from capsule 200 in an expanded configuration (see FIG. 2).

Substrate 320 of expanding band 300 may comprise a single layer of one or more materials, multiple layers of the same material, or multiple layers of different materials with different properties. In certain embodiments, substrate 320 is composed of a shape-memory material, such as a shape memory polymer, configured to expand or uncoil to the expanded configuration in response to changes in moisture, pH, light, chemical concentrations, or other environmental factors. Substrate 320 may be composed of a resilient material (e.g., flexible and spring-like but also rigid) that is forced and held into the loaded configuration by the capsule 200 (or by a dissolvable wrapper or band within the capsule 200) but springs radially outward to the expanded configuration when capsule 200 degrades past a predetermined point. The resilient material is adapted to dissolve or degrade following delivery of the drug through the GI wall. Exemplary polymers for substrate 320 include at least one bioresorbable/biodegradable polymer such as polyglycolic acid, polylactic acid, polycaprolactone, or copolymers and blends thereof that may include polyethylene glycol. In other embodiments, substrate 320 is composed of a thermoplastic polyurethane. In still other embodiments, substrate 320 may be comprised of polymethylmethacrylates, cellulose esters, polyvinyl derivatives, or other biologically inert/safe materials. Substrate 320 of expanding band 300 may be die cut, laser cut, extruded, cast, or formed through other standard polymer shaping processes as is known in the art.

Interfacing surface 310 of expanding band 300 faces radially outward to interface with the patient's GI tract, as described further below. In the illustrated embodiment, interfacing surface 310 is located on an exterior surface of substrate 320. Interfacing surface 310 and substrate 320 may be the same piece, wherein the interfacing surface 310 is the exterior surface of the substrate 320. In another embodiment, interfacing surface 310 is a distinct layer (e.g., coating) that is applied to an exterior surface of substrate 320.

In some embodiments, an additional layer comprising swellable material is disposed or coated on the substrate 320. The swellable material, which may include a hydrogel for example, is adapted to swell upon interaction with fluids, such as the fluids within the GI tract. The swelling of the layer provides additional deployment force to the band 300 to further facilitate uncoiling of the band 300. In one embodiment, the additional layer of swellable material is disposed on the interior surface of the substrate 320, i.e., the substrate surface facing radially inwardly.

Drug 350 of expanding band 300 may be disposed on interfacing surface 310 in solid form. In an exemplary embodiment, the drug 350 is a compound that typically has less efficacy when taken through standard oral delivery and digestion, such as a peptide or protein, like an insulin for example. In an illustrative embodiment, drug 350 includes one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, combined GIP/GLP-1 agonists such as tirzepatide, oxyntomodulin analogs, oxyntomodulin derivatives, therapeutic antibodies, and other suitable therapeutic agents. Drug 350 may also include a vaccine or gene-based drug. In other embodiments, drug 350 may be any biologically active compound to be administered to the patient. Drug 350 may be applied onto or with interfacing surface 310 by dip-coating, spray-coating, painting, or another suitable application technique and dried to its solid form.

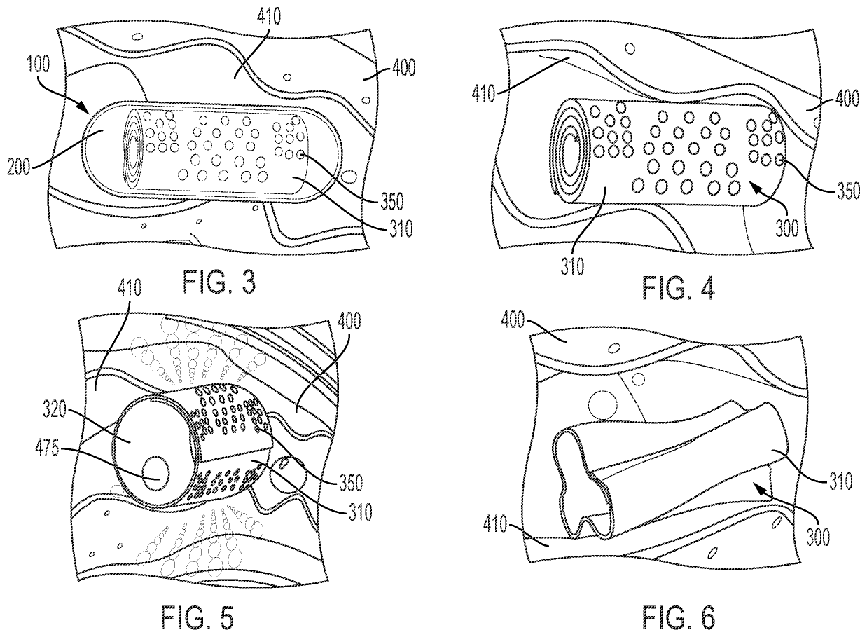

Referring to FIGS. 3-6, in the illustrated embodiment drug delivery device 100 is illustrated activating within a GI tract 400 of a patient. The drug delivery device 100 is administered orally to the patient in the loaded configuration, and then drug delivery device 100 travels into the GI tract 400 (FIG. 3). In an exemplary embodiment, capsule 200 is configured to degrade after entering the small intestine, thereby releasing expanding band 300 in a released configuration (FIG. 4). Once released, the expanding band 300 expands and partially uncoils through the inherent structural properties (e.g., shape-memory properties) of the substrate 320 to the expanded configuration. Expanding band 300 expands and contacts the interior 410 of GI tract 400, while opening to allow space for the passage of GI particulates 475. In this expanded configuration, interfacing surface 310 interfaces with the interior 410 of GI tract 400 and drug 350 is pressed against interior 410 of GI tract 400 (FIG. 5). In the expanded configuration, drug 350 is capable of diffusing through interior 410 of GI tract 400 and into the bloodstream of the patient. After a period of time, expanding band 300 degrades by dissolving or otherwise breaking down in GI tract 400. The degraded band 300 loses structural integrity, collapses from its expanded configuration to a collapsed configuration, and can pass through the GI tract 400 (FIG. 6).

The interfacing surface 310 of expanding band 300 may be coated with a mucosal adhesive to promote adhesion of interfacing surface 310 to the mucosal layer of interior 410 of GI tract 400 in the expanded configuration. By promoting adhesion, mucosal adhesive would increase the period of time in which the interfacing surface 310 is adhered to interior 410 of GI tract 400. Mucosal adhesive may also protect drug 350 during diffusion across the interior 410 of GI tract 400 by limiting exposure of drug 350 to intestinal fluid. Both the increased adherence time and protection of drug 350 may allow for more drug 350 to be administered. Furthermore, interfacing surface 310 may comprise other chemical additives or coatings to promote diffusion of drug 350 across interior 410 of GI tract 400.

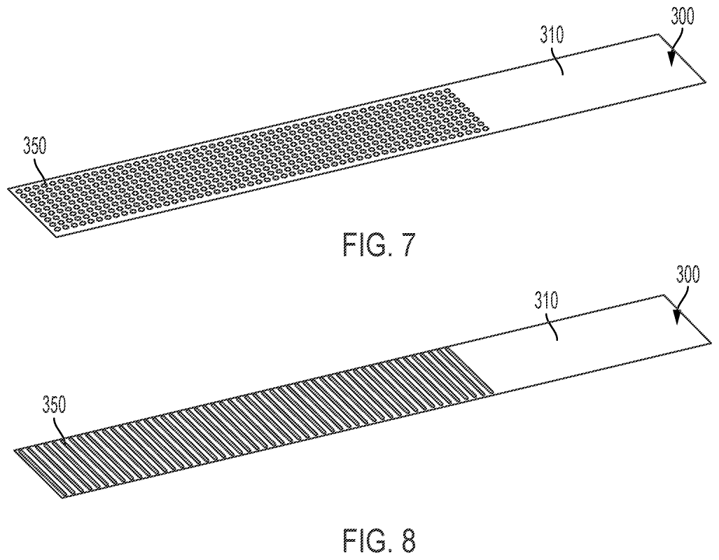

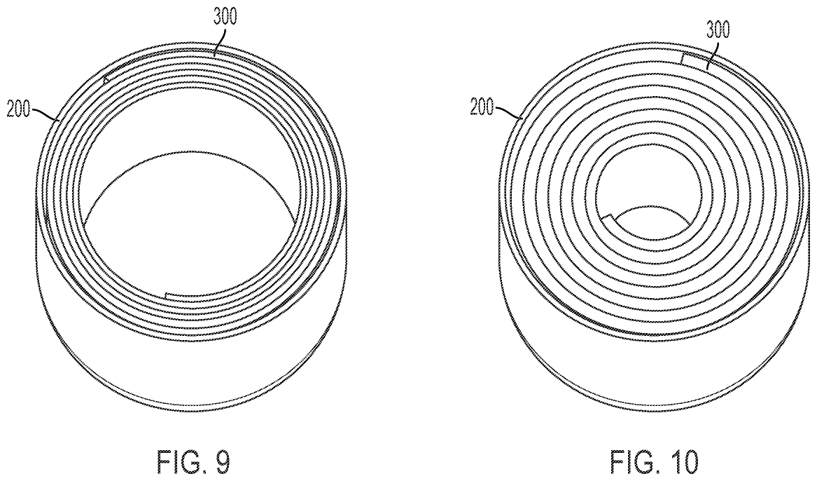

Referring to FIGS. 7-8, drug 350 may be applied to interfacing surface 310 in a variety of patterns. In the illustrated embodiments, drug 350 is applied in a grid dot pattern (FIG. 7) and a linear pattern (FIG. 8). In other embodiments, drug 350 may be a solid coating on the entirety or the majority of interfacing surface 310. Furthermore, drug 350 may be applied in dot patterns that do not follow a standard grid (e.g. diagonal lines, shapes, curves, etc.), or in continuous lines in varying directions and with varying degrees of curvature. In an exemplary embodiment, the drug 350 is applied in a pattern that does not interfere significantly with the structural properties of expanding band 300.

Referring to FIGS. 2 and 9-10, substrate 350 may comprise varying dimensions in various embodiments. In an exemplary embodiment, substrate 350 has a width W between 11 and 17 mm, and a thickness T between 0.1 mm and 0.3 mm. FIGS. 9 and 10 illustrate how altering the thickness T of substrate 350 affects how expanding band 300 fits within capsule 200. In an exemplary embodiment, substrate 350 has a length long enough to accommodate varying GI tract 400 diameters in order to ensure that expanding band 300 is capable of applying approximately equal pressure around the interior 410 of GI tract 400.

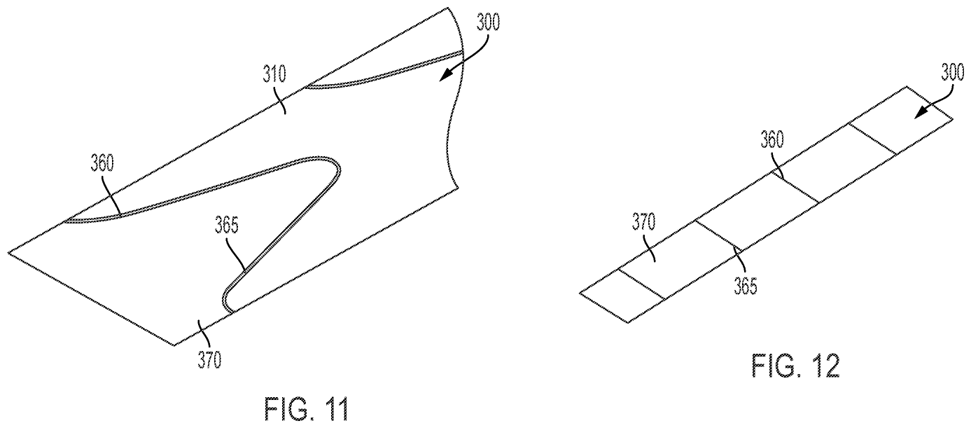

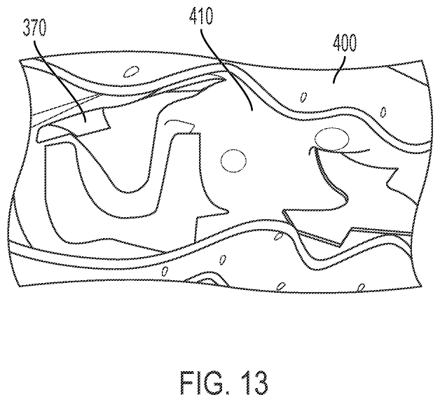

In the illustrated embodiment of FIGS. 1-10, substrate 320 is formed as a single continuous piece composed of one or more of the materials described herein. Referring now to FIGS. 11-13, substrate 320 may alternatively comprise a plurality of substrate segments or pieces 370 coupled together along borders 365 to form substrate 320. The interfacing surface 310 is configured to span across the plurality of substrate pieces 370, such that the substrate pieces 370 cooperate to define the interfacing surface 310. Each individual substrate piece 370 may comprise various shapes. In the illustrated embodiments, substrate pieces 370 comprise curved chevron-shaped segments with curved, non-linear borders 365 (FIG. 11) and rectangular segments with linear borders 365 (FIG. 12). In other embodiments, substrate pieces 370 may be any shape with curved or straight edges and may comprise multiple different shapes in a singular embodiment (e.g. a tessellation). In an exemplary embodiment, substrate pieces 370 are shaped to reduce stress concentration when the expanding band 300 is coiled. In the illustrated embodiment, substrate pieces 370 are coupled together through adhesive 360 applied along the borders 365. Adhesive 360 may be composed of the same materials as substrate pieces 370, or may be composed of a different material, such as an epoxy, resin, gel, glue, or any other coupling means. In an exemplary embodiment, adhesive 360 is composed of a degradable material, such that adhesive 360 may degrade resulting in the degradation of substrate 320 via the separation of substrate pieces 370. Accordingly, expanding band 300 may pass more easily through GI tract 400 (FIG. 13).

In an exemplary embodiment, drug delivery device 100 further comprises a wireless communication device configured to send and/or receive signals to/from a wireless receiver (not shown). The wireless communication device may be configured to measure or sense biological information within the patient after drug delivery device 100 has been ingested. For example, the wireless receiver is operative to send a signal when the expanding band 300 has expanded, or when expanding band 300 has degraded. Furthermore, the wireless communication device may measure/sense other biological information within the GI tract, such as chemical concentrations, pH, temperature, or other biological information. The wireless receiver may be used by the patient receiving treatment, or by another user such as a physician or caretaker. The wireless communication device and wireless receiver may communicate through RFID, magneto-acoustics, near field communications, ultrasonic waves, Bluetooth technology, or other wireless communication means.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An oral drug delivery device comprising:
a biodegradable capsule; and
an expanding band coiled within the biodegradable capsule, the expanding band comprising:
a substrate;
an interfacing surface on the substrate; and
a solid drug applied to the interfacing surface;
wherein the expanding band is configured to expand after the biodegradable capsule has at least partially degraded, the interfacing surface is configured to interface with an interior of a gastrointestinal tract of a patient, and the expanding band is further configured to degrade within the gastrointestinal tract of the patient.

2. The oral drug delivery device of claim 1, wherein the substrate comprises a plurality of substrate pieces.

3. The oral drug delivery device of claim 2, wherein the plurality of substrate pieces are coupled together through a biodegradable adhesive.

4. The oral drug delivery device of claim 2, wherein the plurality of substrate pieces cooperate to define the interfacing surface.

5. The oral drug delivery device of claim 1, wherein the substrate is formed as a single continuous piece.

6. The oral delivery device of claim 1, wherein the substrate is composed of at least one of the following materials: thermoplastic polyurethane, polyglycolic acid, polylactic acid, and polycaprolactone.

7. The oral delivery device of claim 6, wherein the substrate is composed of blend of two or more of polyglycolic acid, polylactic acid, polycaprolactone, and polyethylene glycol.

8. The oral drug delivery device of claim 1, wherein the biodegradable capsule is configured to degrade after a change in pH occurs in an environment surrounding the biodegradable capsule.

9. The oral drug delivery device of claim 1, wherein the substrate has a thickness of about 0.1 mm to about 0.3 mm.

10. The oral drug delivery device of claim 1, wherein the solid drug is applied to the interfacing surface in a repeating pattern of dots.

11. The oral drug delivery device of claim 1, wherein the interfacing surface comprises a mucosal adhesive.

12. The oral drug delivery device of claim 1, wherein the solid drug includes at least one of a peptide and a protein.

13. An oral drug delivery device comprising:
an enteric coating;
a plurality of substrate pieces within the enteric coating, wherein the plurality substrate pieces are coupled together;
an interfacing surface spanning the plurality of substrate pieces, wherein the interfacing surface is configured to interface with an interior of a gastrointestinal tract of a patient; and
a drug applied to the interfacing surface, the drug configured to be absorbed through the interior of the gastrointestinal tract of the patient.

14. The oral drug delivery device of claim 13, wherein the plurality of substrate pieces has curved or linear borders.

15. The oral drug delivery device of claim 13, wherein the plurality of substrate pieces is coupled together through an adhesive.

16. The oral drug delivery device of claim 15, wherein at least one of the plurality of substrate pieces and the adhesive is configured to degrade within the gastrointestinal tract of the patient and to pass through the gastrointestinal tract of the patient.

17. The oral drug delivery device of claim 15, wherein at least one of the plurality of substrate pieces and the adhesive is composed of a polymethylmethacrylate, a cellulose ester, or a polyvinyl derivative.

18. The oral drug delivery device of claim 13, wherein the plurality of substrate pieces is formed from one of laser cutting and die cutting.

19. An oral drug delivery device comprising:
a capsule configured to degrade;
a substrate coiled within the capsule and configured to at least partially uncoil and degrade after the capsule degrades;
an interfacing surface on the substrate, the interfacing surface facing radially outward and configured to interface with an interior of a gastrointestinal tract of a patient; and
a drug applied to the interfacing surface and configured to enter a bloodstream of a patient by diffusing through the interior of the gastrointestinal tract of the patient.

20. The oral drug delivery device of claim 19, wherein the substrate has a width from 11 mm to 18 mm and a thickness from 0.1 mm to 0.3 mm.

21. The oral drug delivery device of claim 19, wherein the interfacing surface comprises a coating on the substrate configured to promote diffusion of the drug through the interior of the gastrointestinal tract of the patient.

22. The oral drug delivery device of claim 21, wherein the substrate comprises a plurality of substrate pieces, the plurality of substrate pieces coupled together through a biodegradable adhesive.

23. The oral drug delivery device of claim 19, wherein the substrate is formed as a single continuous piece.

24. The oral delivery device of claim 19, wherein the substrate is composed of at least one of the following materials: thermoplastic polyurethane, polyglycolic acid, polylactic acid, and polycaprolactone.

25. The oral drug delivery device of claim 19, further comprising a wireless communication device and a wireless receiver, the wireless communication device configured to send biological information to the wireless receiver.

26. The oral drug delivery device of claim 25, wherein the wireless communication device and the wireless receiver communicate through one of RFID, magneto-acoustics, near field communications, ultrasonic waves, and Bluetooth.

27. An oral drug delivery device comprising:
a biodegradable capsule;
an expandable band; and
a solid drug disposed on the expandable band;
wherein the oral drug delivery device has:
a loaded configuration in which the expandable band is coiled within the capsule;
an expanded configuration in which the expandable band at least partially uncoils upon degradation of the capsule to place the solid drug in contact with a patient; and
a collapsed configuration in which the expandable band degrades to pass through the patient.

28. The oral drug delivery device of claim 27, wherein the expandable band is formed as a single continuous piece.

29. The oral delivery device of claim 27, wherein the expandable band is composed of at least one of the following materials: thermoplastic polyurethane, polyglycolic acid, polylactic acid, and polycaprolactone.

30. A method for delivering a drug to a patient through a drug delivery device comprising:
administering the drug delivery device orally to the patient, the drug delivery device including a capsule, an expandable band coiled within the capsule, and a solid drug disposed on an interfacing surface of the expandable band;
degrading the capsule within a small intestine of the patient;
uncoiling the expandable band to engage a wall of the small intestine with the interfacing surface to place the solid drug in contact with the small intestine; and
delivering the solid drug to the patient through the small intestine.

31. The method of claim 30, further comprising degrading the expandable band following the delivering step to collapse the expandable band.
